(19) United States
(12) Patent Application Publication
(10) Pub. No.: US 2005/0106701 A1
(43) Pub. Date: May 19, 2005

(54) Methods for the purification of defensins

(75) Inventor: Luc Guerrier, Versailles (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Ciphergen Biosystems, Inc., Fremont, CA (US)

(21) Appl. No.: 10/769,294

(22) Filed: Jan. 29, 2004

Related U.S. Application Data
(60) Provisional application No. 60/523,153, filed on Nov. 17, 2003.

(51) Int. Cl. C12N 9/64; C07K 14/47

(52) U.S. Cl. 435/226; 530/350

(57) ABSTRACT

The present invention provides, inter alia, methods for the isolation and purification of defensin polypeptides from white blood cells. More particularly, the present invention provides methods for the extraction and purification of defensin polypeptides, i.e., α-defensins 1, 2 and 3, from intact white blood cells, without the need for nitrogen cavitation (Parr bomb) or mechanical disruption, centrifugation and gel filtration.

### CROSS-REFERENCE TO RELATED APPLICATION

This application claims benefit of U.S. Provisional Application No. 60/523,153, filed Nov. 17, 2003, which application is incorporated herein by reference for all purposes.

### FIELD OF THE INVENTION

The present invention is directed to the field of protein biochemistry.

### BACKGROUND OF THE INVENTION

The Human Immunodeficiency Virus (HIV) infects millions of people globally. Cases are reported from nearly every country amounting to 40 million adults and children living with HIV/AIDS worldwide. In 2001, 5 million people were newly infected with HIV, and there were 3 million adult and child deaths due to HIV/AIDS. A full third of those people living with AIDS are aged 15-24 (World Health Organization, 2001). HIV/AIDS treatments exist, however, the drugs currently used in treatment modalities exhibit numerous side effects, ranging from headaches, diarrhea, fatigue, nausea, tingling sensations, abdominal pain, and reduced appetite to elevated kidney and liver functions. Furthermore, the drugs currently used to treat HIV/AIDS require prolonged treatments that often induce drug resistance and do not result in complete eradication of the virus from the body.

It has been known since 1986 that CD8⁺ T lymphocytes from certain HIV-1 infected individuals who are immunologically stable secrete a soluble factor, termed CAF, that suppresses HIV-1 replication. However, until very recently, the identity of CAF remained elusive despite an extensive search. By means of SELDI (ProteinChip® technology, Ciphergen Biosystems, Inc. (Fremont, Calif.)), a cluster of proteins was recently identified that was secreted when CD8⁺ T cells from long-term nonprogressors with HIV-1 infection were stimulated (see, Zhang et al., Science, 298:995-1000 (2002), and U.S. patent application Ser. No. 10/452,763, which was filed May 30, 2003, the teachings of which are incorporated herein by reference). These proteins were identified as α-defensin 1, 2 and 3 on the basis of specific antibody recognition and amino acid sequencing. CAF activity was eliminated or neutralized by an antibody specific for human α-defensins. Synthetic and purified preparations of α-defensins also inhibited the replication of HIV-1 isolates in vitro. Taken together, these results indicate that α-defensin 1, 2, and 3 collectively account for much of the anti-HIV-1 activity of CAF that is not attributable to β-chemokines. As such, methods and compositions employing defensins as anti-viral agents have now been developed (see, U.S. patent application Ser. No. 10/452,763, which was filed May 30, 2003).

In addition to the above recently identified anti-viral activity (e.g., anti-HIV activity), Ganz et al. and others have previously reported the antimicrobial activity of defensin polypeptides. In particular, defensin polypeptides, such as α-defensins 1, 2 and 3, have previously been known to have anti-bacterial and anti-fungal activities (see, e.g., Meth. Enzymology, 236:160-172 (1994), and J. Clin. Invest., 76:1427-1435 (1985)). In this connection, Ganz et al. have previous reported the extraction of α-defensins 1, 2 and 3 (or, alternatively, human neutrophil peptides 1, 2 and 3 (collectively HNP 1-3)) on a small scale (mg α-defensins 1, 2 and 3) from primary granules (azurophiles) of neutrophiles (see, Meth. Enzymology, 236:160-172 (1994)). As set forth in FIG. 1, in the Ganz et al. method, any red cells present in the granulocyte mixture are lysed (hypotonic shock), and the fraction of leukocytes, which has been enriched by plasma-pheresis (granulopak), is isolated by low-speed centrifugation. Thereafter, lysis of the leukocytes is carried out by nitrogen (N₂) cavitation (Parr bomb) to release the granules, which are then isolated by high-speed centrifugation. A crude extract of α-defensins 1, 2 and 3 is obtained by overnight extraction of the granules using 5% acetic acid. Following their extraction, α-defensins 1, 2 and 3, which are basic and hydrophobic polypeptides, are purified from other proteins by size-exclusion chromatography using non-specific retention in two column volumes of α-defensins 1, 2 and 3. Unfortunately, a high dilution of α-defensins 1, 2 and 3 results from this size-exclusion step. Cation exchange chromatrography as well as reverse phase chromatography have also been used to purify α-defensins 1, 2 and 3 from other blood proteins (see, J. Clin. Invest., 76:1427-1435 (1985)).

Although the foregoing method has been used to isolate α-defensins 1, 2 and 3, it has a number of disadvantages. First, it requires the use of a number of centrifugation steps, which can be difficult, especially on a large-scale. Second, it requires the use of gel filtration which is time-consuming and results is high dilution of the α-defensins 1, 2 and 3. Finally, is requires the use of N₂ cavitation (Parr bomb), which results in the disruption of the white cells and is difficult to employ on a large-scale.

In view of the foregoing, there exists a need in the art for methods of isolating α-defensins 1, 2 and 3 from white blood cells that overcome the disadvantages associated with the previously used methods. More particularly, it would be advantageous to have methods of isolating α-defensins 1, 2 and 3 from white blood cells that do not require the use of nitrogen cavitation, centrifugation and gel filtration. Quite surprisingly, the present invention provides such methods.

### SUMMARY OF THE INVENTION

The present invention provides novel methods for the isolation and purification of human defensin polypeptides from white blood cells. The extraction methods of the present invention overcome the disadvantages associated with previously used methods by extracting the defensin polypeptides directly from intact white cells without the need to disrupt the white cells by nitrogen cavitation or some other mechanical means (e.g., homogenization) and without the need for centrifugation. In addition, the purification methods of the present invention overcome the disadvantages associated with previously used methods by purifying defensin polypeptide using column chromatography without the need for gel filtration, which results in the problematic dilution of the defensin polypeptides and is quite time-consuming.

In one aspect, the present invention provides a method for isolating a defensin polypeptide from a white blood cell, the method comprising: (a) contacting the white blood cell with an acid solution to release the defensin polypeptide from the white blood cell into the acid solution; and (b) isolating the defensin polypeptide from the acid solution. Advantageously, this method does not require any white cell disruption, which makes the methods simpler to carry out and avoids the risk of interaction with nucleic acids or other intra-cellular components that can affect defensin polypeptide recovery. Depending on the source of the white blood cells and the extent to which they have been isolated, the white cells can be provided in the form of, for example, a buffy coat, a leukophoresis pack, etc. In addition, the white cells can be isolated granulocytes. Preferably, the white blood cells are provided in the form of buffy coats, which are readily available from a number of different sources. At last, the methods of the present invention can be applied to frozen white blood cells, wherein good results in terms of yield and purity have been obtained.

In one embodiment, the acid has a pKa of less than about 5.0. In another embodiment, the acid has a pKa of less than about 2.5. Exemplary acids suitable for use in the methods of the present invention include, but are not limited to, acetic acid, trifluoroacetic acid, trichloroacetic acid, dichloroacetic acid, fluoroacetic acid, chloroacetic acid, formic acid, hydrochloric acid and sulfuric acid. In certain preferred embodiments, the acid is acetic acid, whereas in other preferred embodiments, the acid is trifluoroacetic acid.

In some embodiments, the acid solution further comprises a water-miscible organic solvent. Exemplary water-miscible organic solvents include, but are not limited to, DMF, DMSO, acetone, methanol, ethanol, isopropanol and acetonitrile. In preferred embodiments, the water-miscible organic solvent is acetonitrile.

In other embodiments, the white cells are contacted with a hypotonic buffer or water to ensure lysis of any contaminating red cells. Typically, the hypotonic lysis of the red cells is carried out prior to contacting the white bloods cells with the acid solution. This step may be repeated to achieve complete removal of contaminating red cells.

In preferred embodiments, the methods of the present invention are advantageously carried out on a filter. Suitable filters includes those having a cut-off such that the white blood cells are retained on the filter and a depth sufficient to hold the desired amount of white blood cells. When a filter in employed, the white bloods cells are placed on the filter prior to contacting the white blood cells with the acid solution to extract the defensin polypeptides. Typically, the white cells are placed on the filter, the filter is washed with water or a hypotonic buffer to remove contaminating red cells and, thereafter, the white blood cells are contacted with the acid solution to extract the defensin polypeptide from the white blood cells.

Once the crude defensin polypeptide extract has been obtained, the defensin polypeptide can be purified using a number of different methods. In preferred embodiments, column chromatography is used to obtain purified defensin polypeptide. Suitable sorbents for use in column chromatography include cation exchange sorbents as well as hydrophobic sorbents.

Other features, objects and advantages of the invention and its preferred embodiments will become apparent from the detailed description, examples and claims that follow.

### BRIEF DESCRIPTION OF THE DRAWINGS

FIG. 1 illustrates the conventional process previously used to isolate α-defensins 1, 2 and 3 from neutrophiles.

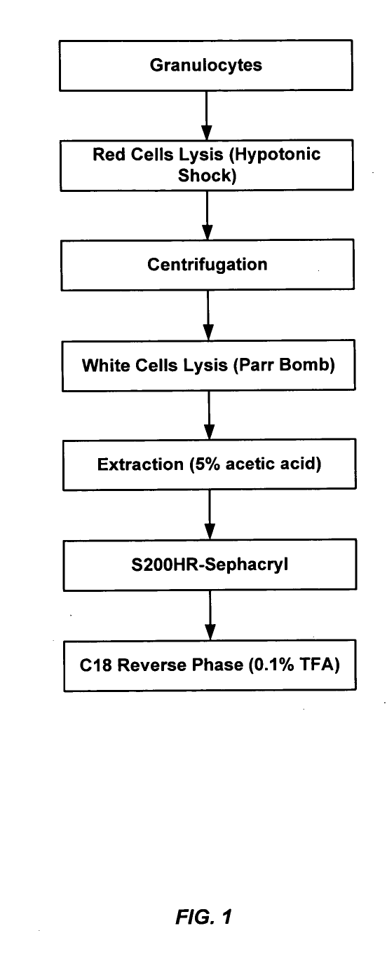

FIG. 2 illustrates an exemplary method of the present invention, wherein α-defensins 1, 2 and 3 are extracted from intact white cells, without the need for nitrogen cavitation (Parr bomb) or other mechanical disruption of the white blood cells.

FIG. 3 illustrates the primary amino acid sequence of human α-defensins 1, 2, 3 and 4, which are referred to therein as HNP-1, -2, -3 and -4, respectively, as well as the primary amino acid sequences of other related defensins from rabbit, rat, mouse and human.

### DETAILED DESCRIPTION OF THE INVENTION AND PREFERRED EMBODIMENTS

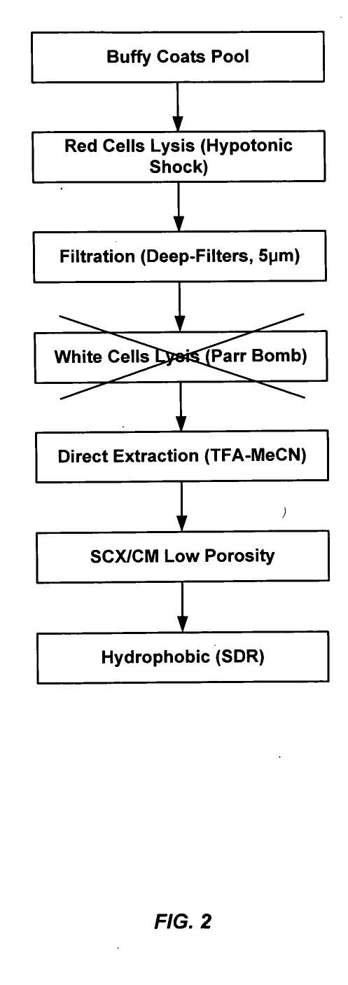

The present invention provides novel methods for the isolation and purification of defensin polypeptides from white blood cells. The methods of the present invention overcome the disadvantages associated with previously used methods by extracting the defensin polypeptides directly from intact white cells without the need to disrupt the white cells by nitrogen cavitation or some other mechanical means (e.g., homogenization) and without the need for centrifugation. FIG. 2 illustrates an exemplary process for the isolation of defensin polypeptides in accordance with the present invention. As illustrated in FIG. 2, the methods of the present invention do not require the use of nitrogen cavitation, centrifugation or gel filtration.

In one embodiment, the present invention provides a method for isolating a defensin polypeptide from a white blood cell, the method comprising: (a) contacting the white blood cell with an acid solution to release the defensin polypeptide from the white blood cell into the acid solution; and (b) isolating the defensin polypeptide from the acid solution.

As used herein, the term “defensin polypeptide(s)” or, alternatively, “defensin(s)” refers to the class of proteins that are well known in the art as anti-bacterial and anti-fungal agents and, more recently, as anti-viral agents (see, U.S. patent application Ser. No. 10/452,763, which was filed May 30, 2003). Defensins have been found and characterized in many animals, including humans, guinea pig, rat, rabbit, macaques, and mice, as well as in plants and insects. The genes encoding the human defensins are thought to be located on a single chromosomal region, 8p21-23 (Kaiser et al., Journal of Leukocyte Biology, 68:779-784 (2000)). Structurally, defensins are cationic molecules with spatially separated hydrophobic and charged regions. The known defensins all share a β sheet structure and 6 or 8 cysteine residues that form intramolecular cysteine disulfide bonds.

As used herein, the term “α-defensin” refers to a polypeptide whose biological activity is characterized as having anti-bacterial activity, anti-fungal activity and anti-viral (such as anti-HIV) activity. The α-defensins are generally less than 100 amino acids longs, usually about 25-35 amino acids long, and are characterized by six cysteine residues in a motif that is conserved between species. The α-defensins also have a net positive charge at physiological pH, generally attributable to positively charged arginines. Accordingly, the term α-defensin encompasses cationic proteins having both high cysteine and high arginine content. In some embodiments, the α-defensins are characterized as having six cysteines and two to four arginines, which are substantially conserved. The cysteines and arginines are dispersed throughout the polypeptide, so that the cysteines provide for the opportunity for extensive crosslinking, intramolecularly and intermolecularly, covalently and noncovalently, and the arginines provide for positive charges throughout the molecule at a wide range of pHs, so as to be highly cationic. For example, in some embodiments, the α-defensins will contain 3 disulfide bonds, formed by the cysteine residues. The α-defensins of the present invention may also form a secondary structure comprising a β hairpin conformation, such as a β sheet, e.g., a triple-stranded antiparallel β sheet (Mandal et al., J Peptide Research, 59:95-104 (2002)).

α-defensins are expressed in various sites throughout the body. However, α-defensins 1-4 are predominantly found in the polymorphonuclear neutrophils, whereas the α-defensins 5-6 are highly expressed in the secretory granules of Paneth cells in the small intestine. Human α-defensins 1-3 have now been identified as being secreted by CD8⁺ T cells. Three of the defensins, i.e., α-defensins-1-3, differ by only one amino acid at the N-terminus (Linzmeier et al., FEBS LETT., 321(2-3):267-273 (1993); Palfree et al., Mol. Endocrin., 7(2):199-205 (1993); Mallow et al., J. Biol. Chem., 271(8):4038-4045; (1996); Mars et al., J. Biol. Chem., 270(51):30371-30376; and Quayle et al., Am. J. Pathol., 152(5): 1247-1258).

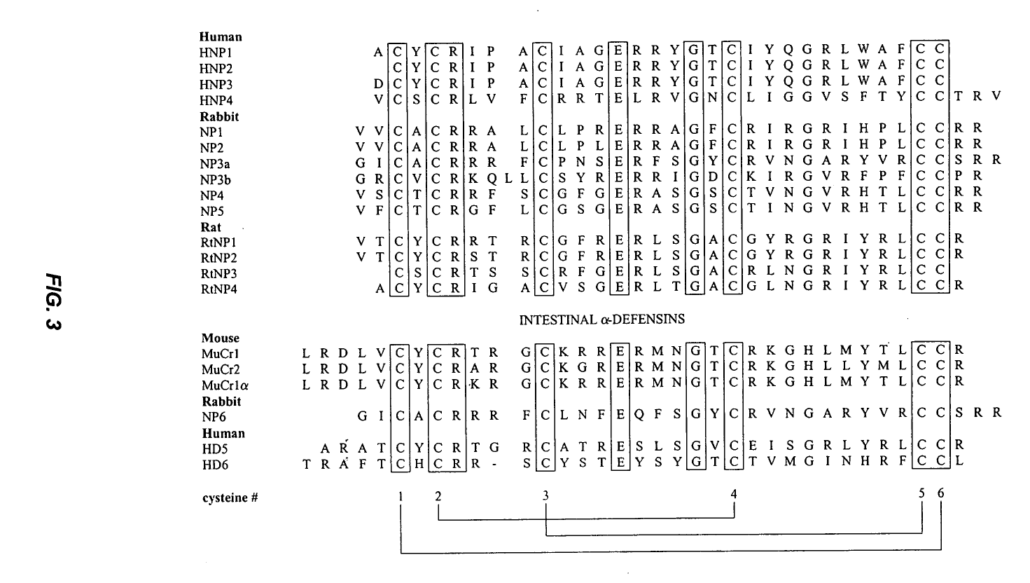

For the purposes of the present invention, the term “defensin polypeptide” or “defensin” include α-defensins and, in particular, α-defensins-1, 2, and 3. Using the methods of the present invention, a mixture of α-defensins-1, 2, and 3 is obtained from intact, non-disrupted white blood cells. It is thought that some α-defensin-4 may be present in the mixture, but to a much lesser extent. The primary amino acid sequence of these α-defensins are illustrated in FIG. 3. It is noted that although the singular form of the term “defenin polypeptide” is used herein, this term is intended to cover the mixture of α-defensins that is isolated from human white cells, i.e., a mixture of α-defensins-1, 2, and 3, using the methods of the present invention.

In the methods of the present invention, the defensin polypeptides are isolated or extracted from intact white blood cells. As used herein, the term “white blood cell(s)” (or, alternatively, “white cell(s)” or “leukocyte(s)” or “white corpuscle”) refers to any of various blood cells that have a nucleus and cytoplasm, that separate into a thin white layer when whole blood is centrifuged, and that help protect the body from infection and disease. White blood cells, as used herein, include neutrophils, eosinophils, basophils, lymphocytes and monocytes. The term “intact” or “intact white blood cell,” as used herein, refers to white blood cells, wherein the cells are not disrupted. More particular, intact white blood cells refer to white blood cells, wherein the nuclear and cytoplasm membranes are not disrupted or broken. It will be readily apparent to those of skill in the art that there are a number of ways to assess the morphology of a white blood cell and to determine whether or not a white blood cell is intact, i.e., not disrupted or broken. For instance, phase-contrast microscopy can be readily used to determine whether or not the white blood cells are intact. Again, one of the advantages of the methods of the present invention is that the defensins can be isolated or extracted directly from intact white blood cells, without the need for cell disruption by N₂ cavitation (Parr bomb) or some other mechanical means. In addition to being cumbersome procedures that are not readily scalable, the disruption of white blood cells by N₂ cavitation or homogenization introduces the risk of nucleic acids and other intra-cellular compounds interacting with defensin polypeptides, and impacting the recovery of the defensin polypeptides.

The white blood cells can be provided in a number of different forms. In one embodiment, the white blood cells are provided in a buffy coat, which is the layer of white blood cells formed when blood is centrifuged. In another embodiment, the white blood cells are provided in a leukaphoresis pack. In yet another embodiment, the white blood cells are granulocytes. It will be readily apparent to those of skill in the art that the white blood cells can be fresh or frozen and, if frozen, the white blood cells are allowed to reach room temperature prior to isolation of the defensin polypeptides.

In the methods of the present invention, the defensin polypeptides are isolated or extracted from intact white blood cells by contacting the white blood cells with an acid solution. Typically, the acid solution is an aqueous acid solution. Suitable acids include those having a pKa of less than about 5.0 and, more preferably, less than about 2.5. Examples of suitable acids include, but are not limited to, acetic acid, trifluoroacetic acid, trichloroacetic acid, dichloroacetic acid, fluoroacetic acid, chloroacetic acid, formic acid, hydrochloric acid and sulfuric acid. In a preferred embodiment, the acid is acetic acid. In another preferred embodiment, the acid is trifluoroacetic acid. Typically, the acid solution is a 0.05% to about 15% acid solution and, more preferably, a 0.1% to about 5% acid solution. As such, in one preferred embodiment, the acid solution is 0.1% trifluoroacetic acid. In another preferred embodiment, the acid solution is 5% acetic acid.

In certain embodiments of the present invention, the acid solution further comprises a water-miscible organic solvent. It has been found that the use of a water-miscible organic solvent assists with and increases the defensin extraction. In fact, it has been found that the combination of acid and solvent quite effectively extracts defensins from the white cells, while leaving in place cell debris and large proteins. Suitable water-miscible organic solvents include, but are not limited to, DMF, DMSO, acetone, methanol, ethanol, isopropanol and acetonitrile. The acid solution typically comprises from about 5% to about 70% of the water-miscible organic solvent and, more preferably, from about 15% to about 35%. In a presently preferred embodiment, the water-miscible organic solvent is acetonitrile. In a preferred embodiment, the acid solution comprises 25% acetonitrile.

The white blood cells are contacted with the acid solution for a period of time sufficient to extract the defensin polypeptides. Quite surprisingly, it has been found that a direct extraction of the defensin polypeptides can be efficiently achieved in high yields in just a few hours. As such, the white blood cells are typically contacted with the acid solution for a period of time ranging from about 1 hour to about 10 hours and, more preferably, from about 2 hours to about 5 hours. It will be readily apparent to those of skill that the extract can be monitored during the contacting step to identify the appropriate portions or fractions containing the defensin polypeptides.

Prior to contacting the white blood cells with the acid solution, it may be desirable to rid the white blood cells of any contaminating erythrocytes, i.e., red blood cells, by hypotonic lysis. The hypotonic lysis is readily carried out by contacting the white blood cells with a hypotonic buffer, such as PBS, or with water, such as deionized water, both of which are preferably used cold. Contaminating red cells are readily lysed by adding ice-cold water to the white blood cells for a period of about 45 to 60 seconds, followed by the recovery of isotonicity (PBS). This red cell lysis step can be repeated one or more times, if necessary.

In another embodiment, the methods of the present invention further comprise purifying the defensin polypeptide such that it is “substantially pure.” As used herein, the term “substantially pure” describes a defensin polypeptide that has been separated from components that naturally accompany it. Typically, a compound is substantially pure when at least 10%, more preferably at least 20%, more preferably at least 50%, more preferably at least 60%, more preferably at least 75%, more preferably at least 90% and most preferably at least 99% of the total material (by volume, by wet or dry weight or by mole percent or mole fraction) in a sample is the defensin polypeptide. Purity can be measured by any appropriate method, such as by column chromatography, gel electrophoresis or HPLC analysis. A defensin polypeptide is also substantially purified when it is essentially free of naturally associated components, or when it is separated from the native contaminants that accompany it in its natural state.

It will be readily apparent to those of skill in the art that a number of different purification techniques can be used to purify the defensin polypeptide from the crude extract. For example, the defensin polypeptides can be purified using chromatographic procedures, such as reverse phase HPLC, gel permeation, ion exchange, e.g., cation exchange, size exclusion, affinity, partition, or countercurrent distribution. Methods of purifying defensins are described in, for example, Raj et al., Biochem. J., 347:633-641 (2000)), and Ganz et al., J. Clin. Invest., 76:1427-1435 (1985).

In a preferred embodiment, the defensin polypeptide is purified using column chromatography. As is known to those in the art, column chromatography employs a stationary phase and a mobile phase. For the purification of defensin polypeptides, the stationary phase typically is a sorbent that has low exclusion limits and complementary properties suitable for the defensins. The use of low exclusion limit chromatographic sorbents is advantageous in that such sorbents discriminate between large and small molecular species and avoid the use of gel filtration materials, which have a low productivity since the load must not exceed 1% of the column volume in order to achieve a proper separation.

As such, in one embodiment, the stationary phase is a cation exchange sorbent (or, alternatively, resin). Typically, the cation exchange sorbent is a porous cation exchange resin having a low exclusion limit. Exemplar cation exchange groups suitable for use include, but are not limited to, carboxylates, sulfonates, sulfates, phosphates and phsophonates Those of skill in the art will know of other cation exchange groups suitable for use in cation exchange resins. In another embodiment, the stationary phase is a hydrophobic sorbent. Typically, the hydrophobic sorbent is a porous hydrophobic resin having a low exclusion limit. Exemplar hydrophobic groups include, but are not limited to, linear C₃ to C₁₈ hydrocarbon chains (such as linear or straight chain —(CH₂)ₙ—CH₃ groups, wherein n is from about 3 to about 18), branched C₃ to C₁₈ hydrocarbon chains (such as branched —(CH₂)ₙ—CH₃ groups, wherein n is from about 3 to about 18), aryls (such as phenyls) that are optionally substituted with non-ionic groups and alkaryls (such as C₃ to C₈ alkylphenyls) that are optionally substituted with non-ionic groups Those of skill in the are will know of other hydrophobic groups suitable for use in hydrophobic resins. Again, both the cation exchange resins and the hydrophobic resins have low exclusion limits. In preferred embodiments, the exclusion limits range from about 5,000 daltons to about 20,000 daltons. In more preferred embodiments, the exclusion limit of the cation exchange and hydrophobic resins is about 5,000 daltons.

Numerous cation exchange and hydrophobic sorbents suitable for use in the purification methods of the present invention are commercially available from a variety of different sources. In a preferred embodiment, the cation exchange sorbent is a LEX-CM-HyperZ resin (wherein LEX indicates a low-exclusion sorbent), which is commercially available from the BioSepra S.A. Process Division of Ciphergen Biosystems, Inc. (Fremont, Calif.). In a preferred embodiment, the hydrophobic sorbent is a SDR HyperD resin, which is also commercially available from the BioSepra S.A. Process Division of Ciphergen Biosystems, Inc. Importantly, the use of such sorbents allows for the use of high speeds that are incompatible with the previously used gel filtration methods. Again, other cation exchange and hydrophobic sorbents, which are porous and have low-exclusion limits, will be known to those of skill in the art and can be used in the methods of the present invention.

Suitable mobile phases for use with the LEX-CM-HyperZ resin are typically acidic buffers having a pH ranging from about 3 to about 6. Examples of such suitable mobile phases include, but are not limited to, acetonitrile, ethanol, methanol, isopropanol, and the like. In a preferred embodiment, the mobile phase is a sodium acetate buffer and, in a preferred embodiment, the sodium acetate buffer has a pH ranging from about 3.5 to about 5.0. In a more preferred embodiment, the sodium acetate buffer has a pH of about 4.5. Suitable mobile phases for the SDR Hyper D resin include, but are not limited to, acetonitrile, ethanol, methanol, isopropanol, and the like. In a preferred embodiment, the mobile phase further comprises an acid. Suitable acids include, but are not limited to, acetic acid, trifluoroacetic acid, trichloroacetic acid, dichloroacetic acid, fluoroacetic acid, chloroacetic acid, formic acid, hydrochloric acid and sulfuric acid. In a preferred embodiment, the acid is trifluoroacetic acid. Typically, the mobile phase comprises from about 0.05% to about 5% acid and, more preferably, from about 0.1% to about 5% acid. Other mobile phases suitable for use in the methods of the present invention will be readily apparent to those of skill in the art.

In a presently preferred embodiment, the purification step comprises the use of two chromatographic columns, wherein the first chromatographic column employs a cation exchange sorbent and the second chromatographic column employs a hydrophobic sorbent. The cation exchange sorbent interacts readily with the cationic character of the defensin polypeptides, whereas the hydrophobic sorbent interacts readily with the defensin polypeptides due to their high hydrophobicity index.

In another aspect of the present invention, the extraction of defensin polypeptides from white blood cells is carried out on a filter. The use of a filter in the extraction methods of the present invention helps to significantly reduce the processed volumes, thereby avoiding a number of disadvantages associated with the previously used methods. In this embodiment, the white blood cells are first retained or placed on (or, alternatively, injected onto) a filter having a cut-off such that the white blood cells are retained on the filter. In a preferred embodiment, the filter has a cut-off ranging from about 2 μm to 10 μm. In a more preferred embodiment, the filter has a cut-off of about 5 μm. Depending on the amount of white blood cells, the filter should be of a depth sufficient to hold the desired amount of white blood cells. In one embodiment, the filter has a cut-off of about 5 μm and a depth of about 10 inches. Filters suitable for use in the methods of the present invention are commercially available from a number of different suppliers, such as Millipore Corporation (Billerica, Mass., USA).

When a filter is used in the methods of the present, an exemplary embodiment of this method comprises: contacting a filter with, for example, a buffy coat comprising white blood cells, the filter having a cut-off such that the white blood cells present in the buffy coat are retained on the filter; washing the filter with water or a hypotonic buffer to lyse any red cells retained on the filter; contacting the white blood cells retained on the filter with an acid solution to release the defensin polypeptide from the white blood cells into the acid solution; and isolating the defensin polypeptide from the acid solution. Advantageously, in this method the acid solution can be readily re-circulated on the filter. The crude extract can be purified as previously described.

Once purified, the defensin polypeptides obtained using the methods of the present invention can be screened for their anti-bacterial, anti-fungal and anti-viral activities using standard methods known to those of skill in the art and, in turn, used for their desired activity.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### EXAMPLES

This example illustrates the isolation of α-defensins 1, 2 and 3 from white blood cells without the need for nitrogen caviation, centrifugation and gel filtration.

630 ml of buffy coat was diluted with 1890 ml of deionized (DI) water and then with 630 ml of 3.5% sodium chloride to achieve a 3150 ml preparation having a physiological ionic strength. The diluted buffy coat was then filtered through a 10 inch-deep filter (5 μm cut-off from Millipore). This step was performed at a flow rate of about 200 ml/min.

After an extensive washing of the filter with PBS (or, alternatively, with deionized water to achieve complete lysis of red cells), a direct extraction of HNP123 was efficiently achieved with good yield in a few hours in situ by re-circulation on the filter of a 25% acetonitrile-0.1% TFA solution in water (1 L). The extract contained HNP123 at a purity of about 20% to about 40% (estimated by HPLC analysis). Concentration of defensins was of about 0.1-0.3 mg/ml.

Purification of HNP 123 from the crude extract was then performed either directly on a strong cation exchanger (S Ceramic HyperD) or on an especially designed low pore/high density cation exchanger (LEX-CM-HyperZ). The crude HNP123 extract is directly injected on S ceramic HyperD) or, alternatively, the crude HNP 123 extract can first be adjusted to a pH of about 4.5 (1 M tris) and then injected into the CM resin. Elution is obtained by salt elution in the presence of 25% acetonitrile. CEX binding capacity was about 30 mg/ml and 10 mg/ml for S HyperD and CM HyperZ, respectively.

The collected HNP 123 fraction was then further purified on SDR HyperD. After adjustment to 10% acetontrile, the collected HNP 123 fraction was directly injected into the column and the pure protein fraction was eluted using a 30 min gradient elution of a solution of acetonitrile containing 0.1% TFA

Acetonitrile was finally removed by roto-evaporation followed by a lyophilization to get a highly purified mixture of HNP123 (more than 90% HPLC purity).

It will be readily apparent to those of skill that the first column can easily be used with alternative solvents, such as ethanol, methanol, isopropanol, etc. The cation exchanger CM, which is a weak cation exchanger, can advantageously be replaced by a low exclusion S cation exchanger, which is a strong cation exchanger. This prevents the adjustment of the crude extract to a pH of about 4 to 5. As with the CM resin, the S resin must also have a low exclusion limit to preserve the ability/property to exclude all large molecular species.

In addition, it will be readily apparent to those of skill that the second column can be used with a large variety of solvents and eluting agents. Suitable solvents include, for example, ethanol, methanol, isopropanol, etc. In addition, the SDR column can be replaced by any other hydrophobic column (C8, C18, etc.) having a low exclusion limit such that it has the ability to exclude all large molecular species.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for isolating a defensin polypeptide from a white blood cell, said method comprising:
(a) contacting said white blood cell with an acid solution to release said defensin polypeptide from said white blood cell into said acid solution; and
(b) isolating said defensin polypeptide from said acid solution.

2. The method in accordance with claim 1, wherein said defensin polypeptide is a human defensin polypeptide.

3. The method in accordance with claim 1, wherein said defensin polypeptide is a mixture of α-defensins 1, 2 and 3.

4. The method in accordance with claim 1, wherein said acid has a pKa of less than about 5.0.

5. The method in accordance with claim 4, wherein said acid has a pKa of less than about 2.5.

6. The method in accordance with claim 1, wherein said acid is a member selected from the group consisting of acetic acid, trifluoroacetic acid, trichloroacetic acid, dichloroacetic acid, fluoroacetic acid, chloroacetic acid, formic acid, hydrochloric acid and sulfuric acid.

7. The method in accordance with claim 1, wherein said acid is acetic acid.

8. The method in accordance with claim 1, wherein said acid is trifluoroacetic acid.

9. The method in accordance with claim 1, wherein said acid solution further comprises a member selected from the group consisting of DMF, DMSO, acetone, methanol, ethanol, isopropanol and acetonitrile.

10. The method in accordance with claim 1, wherein said white blood cell is in a buffy coat.

11. The method in accordance with claim 1, wherein said white blood cell is a granulocyte.

12. The method in accordance with claim 1, wherein said white blood cell is intact.

13. The method in accordance with claim 1, wherein said white blood cell is not disrupted by N₂ cavitation.

14. The method in accordance with claim 1, wherein said white cell is contacted with water prior to step (a).

15. The method in accordance with claim 1, wherein said white cell is contacted with a hypotonic buffer prior to step (a).

16. The method in accordance with claim 11, wherein said buffy coat is contacted with water prior to step (a).

17. The method in accordance with claim 1, wherein said white cell is placed on a filter prior to step (a).

18. The method in accordance with claim 1, wherein said filter has a cut-off ranging from about 2 μm to 10 μm.

19. The method in accordance with claim 18, wherein said filter has a cut-off of about 5 μm.

20. The method in accordance with claim 1, further comprising:
(c) purifying said defensin polypeptide.

21. The method in accordance with claim 20, wherein said defensin polypeptide is purified using column chromatography having a stationary phase and a mobile phase.

22. The method in accordance with claim 21, wherein said stationary phase is a cation exchange resin.

23. The method in accordance with claim 22, wherein said cation exchange resin comprises a cation exchange group selected from the group consisting of carboxylates, sulfonates, sulfates, phosphates and phsophonates.

24. The method in accordance with claim 21, wherein said stationary phase is a hydrophobic resin.

25. The method in accordance with claim 24, wherein said hydrophobic resin comprises a hydrophobic group selected from the group consisting of linear hydrocarbon chains, branched hydrocarbon chains, aryl groups that are optionally substituted with non-ionic groups and alkaryl groups that are optionally substituted with non-ionic groups.

26. The method in accordance with claim 21, wherein said stationary phase has an exclusion limit of between about 5,000 Daltons and 20,000 Daltons.

27. The method in accordance with claim 21, wherein said stationary phase has an exclusion limit of about 5,000 Daltons.

28. The method in accordance with claim 21, wherein said mobile phase is a member selected from the group consisting of sodium acetate, acetonitrile, ethanol, methanol and isopropanol.

29. The method in accordance with claim 28, wherein said mobile phase is sodium acetate having a pH of about 4.5.

30. The method in accordance with claim 28, wherein said mobile phase is acetonitrile.

31. The method in accordance with claim 28, wherein said mobile phase further comprises an acid.

32. The method in accordance with claim 31, wherein said acid is trifluoroacetic acid.

33. The method in accordance with claim 21, wherein said defensin polypeptide is purified using column chromatography wherein said stationary phase is a cation exchange resin.

34. The method in accordance with claim 33, further comprising purifying said defensin polypeptide using column chromatography wherein said stationary phase is a hydrophobic resin.

35. The method in accordance with claim 33 or claim 34, wherein said mobile phase is a member selected from the group consisting of acetonitrile, ethanol, methanol and isopropanol.

36. A method for isolating a defensin polypeptide from a white blood cell, said method comprising:
contacting a filter with a buffy coat comprising white blood cells, said filter having a cut-off such that the white blood cells present in said buffy coat are retained on said filter;
washing said filter with water to lyse any red cells retained on said filter;
contacting the white blood cells retained on said filter with an acid solution to release said defensin polypeptide from the white blood cells into said acid solution; and
isolating said defensin polypeptide from said acid solution.
